SEQUENCE LISTING (11 sequences)

SEQ ID NO: 1 - protein, 14 aa, Clostridium tetani
QYIKANSKFIGITE

SEQ ID NO: 2 - protein, 21 aa, Clostridium tetani
FNNFTVSFWLRVPKVSASHLE

SEQ ID NO: 3 - protein, 21 aa, Plasmodium falciparum
DIEKKIAKMEKASSVFNVVNS

SEQ ID NO: 4 - protein, 15 aa, Rubeola
LSEIKGVIVHRLEGV

SEQ ID NO: 5 - protein, 15 aa, Hepadnaviridae
FFLLTRILTIPQSLD

SEQ ID NO: 6 - protein, 19 aa, Corynebacteriaceae
PVFAGANYAAWAVNVAQVI

SEQ ID NO: 7 - protein, 20 aa, Corynebacteriaceae
VHHNTEEIVAQSIALSSLMV

SEQ ID NO: 8 - protein, 20 aa, Corynebacteriaceae
QSIALSSLMVAQAIPLVGEL

SEQ ID NO: 9 - protein, 20 aa, Corynebacteriaceae
VDIGFAAYNFVESIINLFQV

SEQ ID NO: 10 - protein, 20 aa, Corynebacteriaceae
QGESGHDIKITAENTPLPIA

SEQ ID NO: 11 - protein, 20 aa, Corynebacteriaceae
GVLLPTIPGKLDVNKSKTHI

(19) United States
(12) Patent Application Publication
(10) Pub. No.: US 2007/0048261 A1
(43) Pub. Date: Mar. 1, 2007

(54) Vaccine comprising il-12 or il-23 for treatment of autoimmune diseases

(75) Inventors: Pascal Mettens, Rixensart (BE); Catherine Uyttenhove, Brussels (BE); Jacques Van Snick, Brussels (BE)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GlaxoSmithKline Biologicals S.A., Rixensart (BE)

(21) Appl. No.: 10/582,810

(22) PCT Filed: Dec. 14, 2004
(86) PCT No.: PCT/EP04/14379

§ 371 Date: Jun. 14, 2006

(30) Foreign Application Priority Data
Dec. 16, 2003 (GB) 0329146.5

(51) Int. Cl. A61K 38/20; A61K 39/00; A61K 9/127

(52) U.S. Cl. 424/85.2; 424/450; 424/185.1

(57) ABSTRACT

The present invention provides improved vaccines and immunogenic compositions comprising IL-12 or IL-23, and processes for the preparation of such vaccines and immunogenic compositions.

The present invention relates to improved vaccines and immunogenic compositions, and processes for the preparation of such vaccines and immunogenic compositions.

Interleukin-12 (IL-12) is a heterodimeric cytokine comprising the two subunits P40 and P35. IL-12 is produced mostly by phagocytic cells in response to bacteria, bacterial products, and intracellular parasites, and to some degree by B lymphocytes. In particular, IL-12 is produced by antigen presenting cells and instrumental in induction of TH-1 cell responses. IL-12 induces interferon-γ (IFNγ) from macrophages, natural killer (NK) cells and T lymphocytes, acts as a growth factor for activated NK cells and T lymphocytes, enhances the cytotoxic activity of NK cells, and induces cytotoxic T lymphocyte generation. IL-12 plays a central role in both the induction and magnitude of a primary Th1 response, and is essential to generate and sustain a sufficient number of memory/effector Th1 lymphocytes in vivo to mediate long-term protection against intracellular pathogens.

IL-12 is thought to provide an important contribution to maintaining optimal resistance to intracellular pathogens such as Listeria, mycobacteria, Leishmania major or Toxoplasma. Additionally, individuals with IL-12-receptor deficiency have an increased risk of infection by such pathogens, although resistance to infection seems to increase with age. However, it has been shown that in the absence of IL-12 T cells were still able to mount Th-1 responses to intracellular pathogens that were protective in the absence of IL-10 (Jankovic et al., 2002 Immunity 16:429-439). Moreover, in spite of the increased risk of infection, which was heralded in the first report of IL-12-receptor deficiency in man, individuals with deficient IL-12 function are relatively resistant to infection and resistance seems to increase with age (de Jong et al., 1998 Science 280:1435-1438). One report that examined 41 patients with complete IL-12Rbeta1 deficiency (IL-12Rbeta1 also functions as part of the IL-23 receptor) concluded that human IL-12 is redundant in protective immunity against most microorganisms other than Mycobacteria and Salmonella (Fieschi, et al., 2003. J Exp Med 197:527-535).

IL-12 has been included in vaccine compositions as an adjuvant, to assist in directing the immune response against, for example, tumour antigens contained in the vaccine compositions (WO98/57659).

Interleukin-23 (IL-23) is a heterodimeric cytokine comprising the subunit P40 (common to IL-12) and the subunit P19.

Problems are known to exist with generating an immune response to a self-antigen in vivo.

### STATEMENT OF INVENTION

The present invention provides an immunogenic composition comprising:
(a) an immunogen comprising
   (i) IL-12, IL-23, or a subunit or component thereof; and
   (ii) a carrier;
and (b) an adjuvant comprising one or more of cholesterol; oil-in-water emulsion; oil-in-water emulsion low dose; tocopherol; liposome; QS21; and 3D-MPL.

The present invention is based on the surprising discovery that use of an immunogenic composition as described herein causes an immune response against IL-12 or IL-23 or subunit or component thereof in vivo. Further, the inventors have made the surprising discovery that such an immunogenic composition is extremely effective in the amelioration, treatment or prevention of several diseases.

The present invention further provides a process for the manufacture of an immunogenic composition comprising mixing the immunogen as described herein with an adjuvant as described herein.

The invention further relates to a vaccine composition comprising the immunogenic composition as described herein in combination with a pharmaceutically acceptable excipient, adjuvant or carrier.

The present invention further provides a process for the manufacture of a vaccine composition comprising mixing the immunogenic composition as described herein with a pharmaceutically acceptable excipient, adjuvant or carrier.

The invention further relates to a method of preventing or treating a disease, in particular an autoimmune-implicated disease by administering to an individual at risk of these diseases an immunogenic composition or vaccine composition as described herein.

The invention further provides the use of an immunogenic composition or vaccine composition according to the present invention which is capable of generating an immune response against IL-12 or IL-23, or a subunit or component thereof, in the manufacture of a medicament for the treatment of a disease, in particular an autoimmune-implicated disease.

The invention further comprises a kit comprising an immunogen as described herein, and an adjuvant comprising one or more of cholesterol; oil-in-water emulsion; oil-in-water emulsion low dose; tocopherol; liposome; QS21; and 3 D-MPL.

### DETAILED DECSRIPTION

The immunogenic composition of the present invention is suitably capable of stimulating an immune response to prevent or treat disorders including autoimmune-implicated diseases. The present invention may be used to treat disorders of mammals; for example, the mammal to be treated is human.

Immunogenic Component

An immunogen which forms part of the immunogenic composition according to the present invention is a substance suitably capable of stimulating an immune response. In one embodiment, the immune response is capable of being stimulated in vivo.

IL-12

The term “IL-12” is used herein to mean isolated naturally occurring human or other mammalian interleukin-12, or recombinant human or other mammalian IL-12. By isolated IL-12 is meant IL-12 substantially free of contaminants which may have been present at the beginning of an isolation process. By subunit of IL-12 is meant either of the two peptide subunits, P40 or P35 which comprise IL-12. By component of IL-12 is meant any fragment or epitope of IL-12 or subunit thereof capable of stimulating an immune response against IL-12, fragment or epitope of IL-12 or subunit thereof. In one embodiment of the present invention, the Il-12, subunit or component is human.

IL-23

The term “IL-23” is used herein to mean isolated naturally occurring human or other mammalian interleukin-23, or recombinant human or other mammalian IL-23. By isolated IL-23 is meant IL-23 substantially free of contaminants which may have been present at the beginning of an isolation process. By subunit of IL-23 is meant either of the two peptide subunits, P40 or P19 which comprise IL-23. By component of IL-23 is meant any fragment or epitope of IL-23 or subunit thereof capable of stimulating an immune response against IL-23, fragment or epitope of IL-23 or subunit thereof. In one embodiment of the present invention, the IL-23, subunit or component is human.

In one embodiment of the invention the subunit is P35 of IL-12 or P19 of IL-23. In a further embodiment, the subunit is P40 of IL-12 or IL-23. In a further embodiment, the immunogen comprises at least one surface or discontinuous epitope of one of the subunits of the present invention. The immunogen may comprise at least one surface epitope of P40. The immunogenic composition of the present invention comprising the subunit P40 may be capable of stimulating an immune response against IL-12 or the subunit thereof and or IL-23 or the subunit thereof.

Carrier

Immunogens of the present invention comprise IL-12, IL-23 or a subunit or component thereof as described herein, conjugated to a carrier molecule (for example using chemical conjugation techniques) or fused to a carrier molecule (for example to form a recombinant fusion protein comprising IL-12, IL-23 or a subunit or component thereof and the carrier). The carrier may provide T-cell help for generation of an immune response to the immunogen.

An example of an immunogen which may be used in the present invention is the P40 subunit of either IL-12 or IL-23, conjugated or fused to a carrier protein to provide T-cell help for generation of an immune response to P40.

A non-exhaustive list of carriers which may be used in the present invention includes: Keyhole Limpet Haemocyanin (KLH), serum albumins such as bovine or human serum albumin (BSA or HSA), ovalbumin (OVA), inactivated bacterial toxins such as tetanus toxoid (TT) or diphtheria toxoid (DT), or recombinant fragments thereof (for example, Domain 1 of Fragment C of TT, or the translocation domain of DT), the purified protein derivative of tuberculin (PPD). In an embodiment of the invention in which the carrier protein is of animal-origin, such as KLH or a serum albumin, the carrier protein may be recombinantly derived.

In one embodiment of the invention the carrier may be Protein D from Haemophilus influenzae (EP0594610B1 incorporated herein by reference). Protein D is an IgD-binding protein from Haemophilus influenzae and has been patented by Forsgren (WO 91/18926, granted EP 0 594 610 B1 incorporated herein by reference). In some circumstances, for example in recombinant immunogen expression systems it may be desirable to use fragments of protein D, for example Protein D ⅓^{rd} (comprising the N-terminal 100-110 amino acids of protein D (GB 9717953.5 incorporated herein by reference)).

In one embodiment of the present invention immunogenicity of the immunogen is enhanced by the addition of a “T-cell helper (Th) epitope” or “T-helper epitope”, which is a peptide able to bind to an MHC molecule and stimulate T-cells in an animal species. The T-helper epitope may be a foreign or non-self epitope. T-cell epitopes may be promiscuous epitopes, ie. epitopes that bind to a substantial fraction of MHC class II molecules in an animal species or population (Panina-Bordignon et al, EJI. 1989, 19:2237-2242; Reece et al, JI 1993, 151:6175-6184 incorporated herein by reference).

The immunogenic components of the present invention may, therefore, comprise an immunogen comprising IL-12 or IL-23 or a subunit or component thereof and promiscuous Th epitopes either as chemical conjugates or as purely synthetic peptide constructs. The immunogen may be joined to the Th epitopes via a spacer (e.g., Gly-Gly) at either the N- or C-terminus of the immunogen. In order for the immunogenic components of the present invention to be sufficiently clinically effective, it may be necessary to include several foreign T-cell epitopes. The immunogenic components may comprise 1 or more promiscuous Th epitopes, and in one embodiment may comprise between 2 to 5 Th epitopes.

The Th epitope can consist of a continuous or discontinuous epitope. Th-epitopes that are promiscuous are highly and broadly reactive in animal and human populations with widely divergent MHC types (Partidos et al. (1991) “Immune Responses in Mice Following Immunisation with chimaeric Synthetic Peptides Representing B and T Cell Epitopes of Measles Virus Proteins” J. of Gen. Virol. 72:1293-1299; U.S. Pat. No. 5,759,551). The Th domains that may be used in accordance with the present invention have from about 10 to about 50 amino acids, for example from about 10 to about 30 amino acids. When multiple Th epitopes are present, these may all be the same (ie the epitopes are homologous) or a combination of more than one type of epitope may be used (ie the epitopes are heterogeneous).

Th epitopes include as examples, pathogen derived epitopes such as Hepatitis surface or core (peptide 50-69, Ferrari et al., J. Clin. Invest, 1991, 88, 214-222) antigen Th epitopes, Pertussis toxin Th epitopes, tetanus toxin Th epitopes (such as P2 (EP 0 378 881 B1 incorporated herein by reference) and P30 (WO 96/34888, WO 95/31480, WO 95/26365 incorporated herein by reference), measles virus F protein Th epitopes, Chlamydia trachomatis major outer membrane protein Th epitopes (such as P11, Stagg et al., Immunology, 1993, 79, 1-9), Yersinia invasin, diphtheria toxoid, influenza virus haemagluttinin (HA), and P.falciparum CS antigen.

Other Th epitopes are described in the literature, including: WO 98/23635; Southwood et al., 1998, J. Immunol., 160: 3363-3373; Sinigaglia et al., 1988, Nature, 336: 778-780; Rammensee et al., 1995, Immunogenetics, 41: 4, 178-228; Chicz et al., 1993, J. Exp. Med., 178:27-47; Hammer et al., 1993, Cell 74:197-203; and Falk et al., 1994, Immunogenetics, 39: 230-242, U.S. Pat. No. 5,759,551; Cease et al., 1987, PNAS 84, 4249-4253; Partidos et al., J. Gen. Virol, 1991, 72, 1293-1299; WO 95/26365 and EP 0 752 886 B. The T-cell epitope can also be an artificial sequence such as a Pan D-R peptide “PADRE” (WO 95/07707 incorporated herein by reference). In one embodiment of the present invention, the carrier used is PADRE.

The T-cell epitope may be selected from the group of epitopes that will bind to a number of individuals expressing more than one MHC II molecules in humans. For example, epitopes that are specifically contemplated are P2 and P30 epitopes from TT (Panina-Bordignon Eur. J. Immunol 1989 19 (12) 2237). In one embodiment the heterologous T-cell epitope is P2 or P30 from TT.

The P2 epitope has the sequence QYIKANSKFIGITE (SEQ ID No: 1) and corresponds to amino acids 830-843 of the Tetanus toxin.

The P30 epitope (residues 947-967 of Tetanus Toxin) has the sequence FNNFTVSFWLRVPKVSASHLE (SEQ ID No: 2); the FNNFTV sequence may optionally be deleted.

Other universal T epitopes are derivable from the circumsporozoite protein from Plasmodium falciparum—in particular the region 378-398 having the sequence DIEKKIAKMEKASSVFNVVNS (SEQ ID No: 3) (Alexander J, (1994) Immunity 1 (9), p 751-761).

Another epitope which may be used is derived from Measles virus fusion protein at residue 288-302 having the sequence LSEIKGVIVHRLEGV (SEQ ID No: 4) (Partidos C D, 1990, J. Gen. Virol 71(9) 2099-2105).

Yet another epitope which may be used is derived from hepatitis B virus surface antigen, in particular amino acids, having the sequence FFLLTRILTIPQSLD (SEQ ID No: 5).

Another set of epitopes which may be used is derived from diphtheria toxin. Four of these peptides (amino acids 271-290, 321-340, 331-350, 351-370) map within the T domain of fragment B of the toxin, and the remaining 2 map in the R domain (411-430, 431-450):

| | |
|---|---|
| PVFAGANYAAWAVNVAQVI | (SEQ ID No: 6) |
| VHHNTEEIVAQSIALSSLMV | (SEQ ID No: 7) |
| QSIALSSLMVAQAIPLVGEL | (SEQ ID No: 8) |
| VDIGFAAYNFVESIINLFQV | (SEQ ID No: 9) |
| QGESGHDIKITAENTPLPIA | (SEQ ID No: 10) |
| GVLLPTIPGKLDVNKSKTHI | (SEQ ID No: 11) |

(Raju R., Navaneetham D., Okita D., Diethelm-Okita B., McCormick D., Conti-Fine B. M. (1995) Eur. J. Immunol. 25: 3207-14.)

In one embodiment, the immunogen may be directly conjugated to liposome carriers, which may additionally comprise immunogens capable of providing T-cell help.

The ratio of immunogen to carrier molecules may be in the order of between about 1:10 to about 20:1. Each carrier may carry between about 3 to about 15 molecules of immunogen. In an alternative embodiment, each immunogen may carry between about 3 to about 15 carrier molecules. In an embodiment of the invention in which the carrier is PADRE or a Tetanus peptide, the ratio of immunogen to carrier peptides is between about 1:5 to about 1:10.

Conjugation or Fusion Protein

The immunogen of the present invention may be coupled to the carrier by a method of conjugation well known in the art. Thus, for example, for direct covalent coupling it is possible to utilise a carbodiimide, glutaraldehyde or (N-[γ-maleimidobutyryloxy]succinimide ester, utilising common commercially available heterobifunctional linkers such as CDAP and SPDP (using manufacturers instructions). After the coupling reaction, the conjugate immunogen can easily be isolated and purified by means of a dialysis method, a gel filtration method, a fractionation method etc. Conjugates formed by use of gluteraldehyde or maleimide chemistry may be used in the present invention. In one embodiment, maleimide chemistry may be used.

Alternatively, the immunogen may be fused to the carrier. For example, EP0421635B (incorporated herein by reference) describes the use of chimaeric hepadnavirus core antigen particles to present foreign peptide sequences in a virus-like particle. As such, fusion molecules may comprise immunogen of the present invention presented in chimaeric particles consisting of e.g. hepatitis B core antigen. Alternatively, the recombinant fusion proteins may comprise immunogen and NS1 of the influenza virus. For any recombinantly expressed protein which forms part of the present invention, the nucleic acid which encodes said protein also forms an aspect of the present invention.

The conjugate or fusion protein may be substantially biologically inactive, such that it is substantially unable to signal through IL-12 or IL-23 receptors.

Adjuvant

The vaccine or composition according to the invention comprises an adjuvant or immunostimulant. Adjuvants which may be used include (but are not limited to) those in the following list: detoxified lipid A from any source and non-toxic derivatives of lipid A, saponins and other reagents capable of stimulating a TH1 type response.

It has long been known that enterobacterial lipopolysaccharide (LPS) is a potent stimulator of the immune system, although its use in adjuvants has been curtailed by its toxic effects. A non-toxic derivative of LPS, monophosphoryl lipid A (MPL), produced by removal of the core carbohydrate group and the phosphate from the reducing-end glucosamine, has been described by Ribi et al (1986, Immunology and Immunopharmacology of bacterial endotoxins, Plenum Publ. Corp., NY, p407-419) and has the following structure:

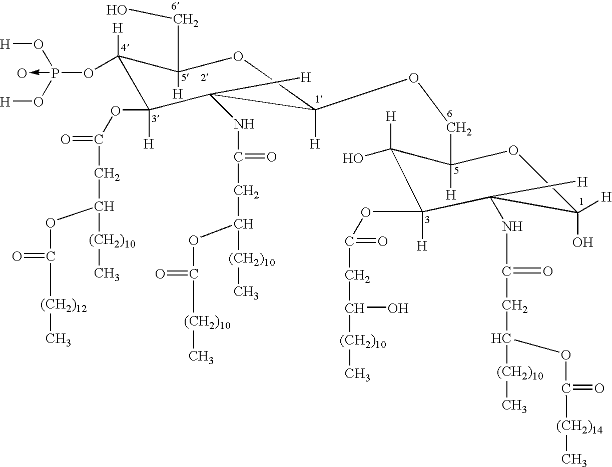

A further detoxified version of MPL results from the removal of the acyl chain from the 3-position of the disaccharide backbone, and is called 3-O-Deacylated monophosphoryl lipid A (3D-MPL). It can be purified and prepared by the methods taught in GB 2122204B, which reference also discloses the preparation of diphosphoryl lipid A, and 3-O-deacylated variants thereof.

One form of 3D-MPL which may be used is in the form of an emulsion having a small particle size less than 0.2 μm in diameter, and its method of manufacture is disclosed in WO 94/21292. Aqueous formulations comprising monophosphoryl lipid A and a surfactant have been described in WO9843670A2.

The bacterial lipopolysaccharide derived adjuvants to be formulated in the compositions of the present invention may be purified and processed from bacterial sources, or alternatively they may be synthetic. For example, purified monophosphoryl lipid A is described in Ribi et al 1986 (supra), and 3-O-Deacylated monophosphoryl or diphosphoryl lipid A derived from Salmonella sp. is described in GB 2220211 and U.S. Pat. No. 4,912,094. Other purified and synthetic lipopolysaccharides have been described (Hilgers et al., 1986, Int. Arch. Allergy. Immunol., 79(4):392-6; Hilgers et al., 1987, Immunology, 60(1):141-6; and EP 0 549 074 B1). A bacterial lipopolysaccharide adjuvant which may be used is 3D-MPL.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

The adjuvant may additionally comprise a saponin, for example QS21. Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Other saponins which have been used in systemic vaccination studies include those derived from other plant species such as Gypsophila and Saponaria (Bomford et al., Vaccine, 10(9):572-577, 1992).

An enhanced system involves the combination of a non-toxic lipid A derivative and a saponin derivative. One system which may be used is the combination of QS21 and 3D-MPL as disclosed in WO 94/00153, or a less reactogenic composition may be used wherein the QS21 is quenched with cholesterol as disclosed in WO 96/33739.

A particularly potent adjuvant formulation which may be used comprises QS21 and 3D-MPL in an oil-in-water emulsion (described in WO 95/17210).

The formulation may additionally comprise an oil-in-water emulsion. In one embodiment of the present invention, the adjuvant consists of an oil-in-water emulsion. Oil-in-water emulsions which may be used are described in PCT application no. WO 95/17210. These may have a high, ratio of squalene:saponin (w/w) of 240:1. Emulsions having a ratio of squalene:QS21 in the range of 1:1 to 200:1, may be used in the present invention. Emulsions having a ratio of squalene:QS21 in the range of substantially 48:1 may also be used in the present invention. This reduction of one of the components has the surprising effect of qualitatively improving the resultant immune response. Using this adjuvant formulation strong Th2-type responses may be maintained, but moreover such formulations elicit an enhanced immune response specifically associated with Th1-type responses, characterised by high IFN-γ, T-cell proliferative and CTL responses.

The present invention also provides a method for producing a vaccine formulation comprising mixing an immunogen and carrier of the present invention together with a pharmaceutically acceptable adjuvant and/or excipient.

An adjuvant suitable for use in the invention is the combination of QS21, 3D-MPL and an oil-in-water emulsion, or the combination of 3D-MPL and QS21 quenched with cholesterol as described above.

The composition of the invention may be delivered by any suitable delivery means and route of administration, suitably by intramuscular injection.

In one aspect of the present invention, the immunogen and carrier of the present invention may be encapsulated into microparticles such as liposomes. Encapsulation within liposomes is described, for example, by Fullerton, U.S. Pat. No. 4,235,877.

Typically, when 3D-MPL is used, the antigen and 3D-MPL are delivered with alum or presented in an oil-in-water emulsion or multiple oil-in-water emulsions. The incorporation of 3D-MPL is advantageous since it is a stimulator of effector T-cell responses.

Accordingly in one embodiment of the present invention there is provided a vaccine comprising an immunogen and carrier as herein described, in combination with 3D-MPL and a vehicle. Typically the vehicle may be an oil-in-water emulsion or alum.

In one embodiment, the adjuvant for use in the present invention may be selected from the group of adjuvants comprising: a monophosphoryl lipid A or derivative thereof such as 3D-MPL, QS21, a mixture of QS21 and cholesterol, and a CpG oligonucleotide. Another adjuvant which may be used comprises a monophosphoryl lipid A or derivative thereof such as 3D-MPL, QS21 and tocopherol in an oil-in-water emulsion. The monophosphoryl lipid A or derivative thereof may be 3D-MPL.

An adjuvant suitable for use in the present invention is a formulation comprising QS21 and an oil-in-water emulsion, wherein the oil-in-water emulsion comprises a metabolisable oil, such as squalene, α-tocopherol and a polysorbate (including polyoxyethylene sorbitan monooleate, TWEEN 80), said emulsions being characterised in that the ratio of the oil:QS21 is in the range of 20:1 to 200:1 (w/w), for example substantially 48:1 (w/w). Such a formulation once combined with an antigen or antigenic preparation is suitable for a broad range of monovalent or polyvalent vaccines. Additionally the oil-in-water emulsion may contain polyoxyethylene sorbitan trioleate (SPAN 85). The oil-in-water emulsion may contain cholesterol.

The ratio of QS21:3D-MPL (w/w) in an embodiment of the present invention may typically be in the order of 1:10 to 10:1; for example 1:5 to 5:1 and often substantially 1:1. A range for optimal synergy may be from 2.5:1 to 1:1 3D MPL:QS21. Typically, the dosages of QS21 and 3D-MPL in a vaccine for human administration will be in the range 1 μg-1000 μg, for example 10 μg-500 μg, for example 10-100 μg per dose. Typically the oil-in-water will comprise from 2 to 10% squalene, from 2 to 10% α-tocopherol and from 0.4 to 2% polyoxyethylene sorbitan monooleate (TWEEN 80). The ratio of squalene: α-tocopherol may be equal or less than 1 as this provides a more stable emulsion. Polyoxyethylene sorbitan trioleate (SPAN 85) may also be present at a level of 0.5-1%. In some cases it may be advantageous that the vaccines of the present invention will further contain a stabiliser, for example other emulsifiers/surfactants, including caprylic acid (Merck index 10th Edition, entry no. 1739), of which Tricaprylin is one embodiment.

Therefore, another embodiment of this invention is a vaccine containing QS21 and an oil-in-water emulsion falling within the desired ratio, which is formulated in the presence of a sterol, for example cholesterol, in order to reduce the local reactogenicity conferred by the QS21. The ratio of the QS21 to cholesterol (w/w), present in a specific embodiment of the present invention, is envisaged to be in the range of 1:1 to 1:20, substantially 1:10.

The emulsions used in PCT application no. WO 95/17210, in particular adjuvants comprising oil-in-water emulsion, MPL and QS21 are adjuvants which may be used in the present invention. It has been observed that formulation of the QS21 into cholesterol containing liposomes may help prevent necrosis occurring at the site of injection. This observation is subject to PCT Application No. PCT/EP96/01464, and the adjuvant disclosed therein, particularly an adjuvant comprising liposome, MPL and QS21 is also a suitable adjuvant for use in the present invention.

In embodiments of the present invention a sterol which may be used is cholesterol. Other sterols which could be used in embodiments of the present invention include β-sitosterol, stigmasterol, ergosterol, ergocalciferol and cholesterol. Sterols are well known in the art. Cholesterol is well known and is, for example, disclosed in the Merck Index, 11th Edn., page 341, as a naturally occurring sterol found in animal fat.

Such preparations are used as vaccine adjuvant systems and once formulated together with antigen or antigenic preparations for potent vaccines. Advantageously they may induce a Th1 response.

The emulsion systems of the present invention may have a small oil droplet size in the sub-micron range. For example the oil droplet sizes will be in the range 120 to 750 nm, for example from 120-600 nm in diameter.

A form of 3 De-O-acylated monophosphoryl lipid A is in the form of an emulsion having a small particle size less than 0.2 μm in diameter.

In one embodiment of the present invention, the adjuvant is SB62′c, an adjuvant comprising an oil-in-water emulsion and a saponin, wherein the oil is a metabolisable oil, and the ratio of the metabolisable oil:saponin (w/w) is in the range of 1:1 to 200:1 (oil-in-water emulsion low dose) described in WO99/11241, the full teaching of which is incorporated herein by reference. In one embodiment, the ratio of the metabolisable oil:saponin (w/w) is substantially 48:1. The saponin may be a QuilA, such as QS21. In one example, the metabolisable oil is squalene. The SB62′c adjuvant composition may further comprise a sterol, for example cholesterol. The SB62′c adjuvant composition may additionally or alternatively further comprise one or more immunomodulators, for example: 3D-MPL and/or α-tocopherol. In an embodiment of SB62′c which comprises 3D-MPL, the ratio of QS21:3D-MPL (w/w) may be from 1:10 to 10:1, for example 1:1 to 1:2.5, or 1:1 to 1:20.

Thus, in one embodiment of the adjuvant SB62′c, the ratio of the metabolisable oil:saponin (w/w) is in the range of 1:1 to 200:1 or is substantially 48:1, the saponin is QS21 and the adjuvant also includes 3D-MPL (oil-in-water emulsion low dose, QS21, 3D-MPL).

In a further embodiment of the present invention, the adjuvant consists of an oil-in-water emulsion comprising a tocol, for example as described in EP0382271. In a further embodiment, the oil-in-water emulsion which may be used comprises α-tocopherol.

In one embodiment, the adjuvant is an adjuvant composition as described herein, presented within a liposome, for example as described in EP822831.

Vaccines

The present invention also provides a vaccine comprising an immunogenic composition as described herein, with a pharmaceutically acceptable excipient, adjuvant or vehicle. The present invention also provides a process for the manufacture of a vaccine composition comprising mixing an immunogenic composition as described herein with appropriate pharmaceutically acceptable vehicles, adjuvants or excipients. Appropriate vehicles and excipients are well known in the art and include for example water or buffers. Vaccine preparation is generally described in Vaccine Design (“The subunit and adjuvant approach” (eds Powell M. F. & Newman M. J.) (1995) Plenum Press New York).

Peptide Synthesis

Peptides used in the present invention can be readily synthesised by solid phase procedures well known in the art. Suitable syntheses may be performed by utilising “T-boc” or “F-moc” procedures. Cyclic peptides can be synthesised by the solid phase procedure employing the well-known “F-moc” procedure and polyamide resin in the fully automated apparatus. Alternatively, those skilled in the art will know the necessary laboratory procedures to perform the process manually. Techniques and procedures for solid phase synthesis are described in ‘Solid Phase Peptide Synthesis: A Practical Approach’ by E. Atherton and R. C. Sheppard, published by IRL at Oxford University Press (1989). Alternatively, the peptides may be produced by recombinant methods, including expressing nucleic acid molecules encoding the mimotopes in a bacterial or mammalian cell line, followed by purification of the expressed mimotope. Techniques for recombinant expression of peptides and proteins are known in the art, and are described in Maniatis, T., Fritsch, E. F. and Sambrook et al., Molecular cloning, a laboratory manual, 2nd Ed.; Cold Spring Harbor Laboratory Press, Cold Spring Harbor, N.Y. (1989).

Nucleic Acids

Also forming part of the present invention are nucleic acids encoding immunogens of the present invention or encoding recombinant fusion proteins comprising the immunogens. In particular isolated nucleic acid molecules which encode an immunogen of the present invention, for example together with a carrier, are provided, which may be used for DNA vaccination. Helpful background information in relation to DNA vaccination is provided in “Donnelly, J et al Annual Rev. Immunol. (1997) 15:617-648, the disclosure of which is included herein in its entirety by way of reference.

In an embodiment of the present invention in which the immunogen is encoded by nucleic acid for use in nucleic acid vaccination, the adjuvant used should be an adjuvant suitable for use in nucleic acid vaccination. Examples of such adjuvants include: synthetic imidazoquinolines such as imiquimod [S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemokine and co-stimulatory molecules as either protein or peptide, this would include pro-inflammatory cytokines such as interferons, particular interferons and GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLA-4 and L-selectin, apoptosis stimulating proteins and peptides such as Fas, (49), synthetic lipid based adjuvants, such as vaxfectin, (Reyes et al., ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A. Other bacterial derived immunostimulating proteins include, Cholera Toxin, E. Coli Toxin and mutant toxoids thereof. Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

In an embodiment of the present invention in which the immunogen is administered in the form of a DNA vaccination, the composition may further comprise a vehicle. For example, the vehicle is a gold bead, or comprises a gold bead. Other vehicles or excipients described herein may also be used. The nucleic acid constructs may be formulated within plasmids for delivery.

Therapeutic Uses

The formulations of the present invention maybe used for both prophylactic and therapeutic purposes. In a further aspect of the present invention there is provided a composition as herein described for use in medicine.

The preparations of the present invention may be used to protect or treat a mammal susceptible to, or suffering from a disease, by means of administering said vaccine via systemic or mucosal route. These administrations may include injection via the intramuscular, intraperitoneal, intradermal or subcutaneous routes; or via mucosal administration to the oral/alimentary, or respiratory tracts.

In one aspect of the present invention there is provided a method of treating a disease, for example a neurological or autoimmune-implicated disorder, by administration of a vaccine according to the present invention. The vaccine of the present invention is useful in the prevention, treatment and/or amelioration of clinical signs associated with neurological diseases such as multiple sclerosis or Guillain-Barré Syndrome, myasthenia gravis; bowel diseases such as Crohn's disease; and autoimmune-implicated diseases including but not limited to systemic lupus erythematosis, rheumatoid arthritis, thyroiditis including Hashimoto's thyroiditis, pernicious anaemia, Addison's disease, diabetes, dermatomyositis, Sjogren's syndrome, multiple sclerosis, Reiter's syndrome, Graves disease and psoriasis. For example, the vaccine of the present invention may be used in the prevention, treatment and/or amelioration of clinical signs associated with one or more of the following conditions: multiple sclerosis; Crohn's disease; thyroiditis; and rheumatoid arthritis.

Dosing Regimen

Vaccines may be delivered in any suitable dosing regime, such as a one, two, three or more dose regimes. Following an initial vaccination, subjects may receive one or several booster immunisation adequately spaced. Such a vaccine formulation may be either a priming or boosting vaccination regime; be administered systemically, for example via the transdermal, subcutaneous or intramuscular routes or applied to a mucosal surface via, for example, intra nasal or oral routes.

It is possible for the vaccine composition to be administered on a once off basis or to be administered repeatedly, for example, between 1 and 7 times, for example between 1 and 4 times, at intervals between about 1 day and about 18 months, for example one month. This may be optionally followed by dosing at regular intervals of between 1 and 12 months for a period up to the remainder of the patient's life. For example, following an initial vaccination, subjects will receive a boost in about 4 weeks, followed by repeated boosts every six months for as long as a risk of infection or disease exists. The immune response to the protein of this invention is enhanced by the use of adjuvant and or an immunostimulant.

In an embodiment of the present invention the patient will receive the antigen in different forms in a prime/boost regime. Thus for example an antigen will be first administered as a DNA based vaccine and then subsequently administered as a protein adjuvant base formulation, or vice versa. Once again, however, this treatment regime will be significantly varied depending upon the size and species of animal concerned, the amount of nucleic acid vaccine and/or protein composition administered, the route of administration, the potency and dose of any adjuvant compounds used and other factors which would be apparent to a skilled veterinary or medical practitioner.

The amount of protein in each vaccine dose is selected as an amount which induces an immunoprotective response without significant, adverse side effects in typical vaccinees. Such amount will vary depending upon which specific immunogen is employed and whether or not the vaccine is adjuvanted. Generally, it is expected that each dose will comprise 1-1000 μg of protein, for example 1-500 μg, for example 1-200 μg, for example 1-100 μg or for example 1-50 μg. An optimal amount for a particular vaccine can be ascertained by standard studies involving observation of antibody titres and other responses in subjects. There can, of course, be individual instances where higher or lower dosage ranges are merited, and such are within the scope of this invention.

The invention is now illustrated by the following non-limiting examples and Figures in which:

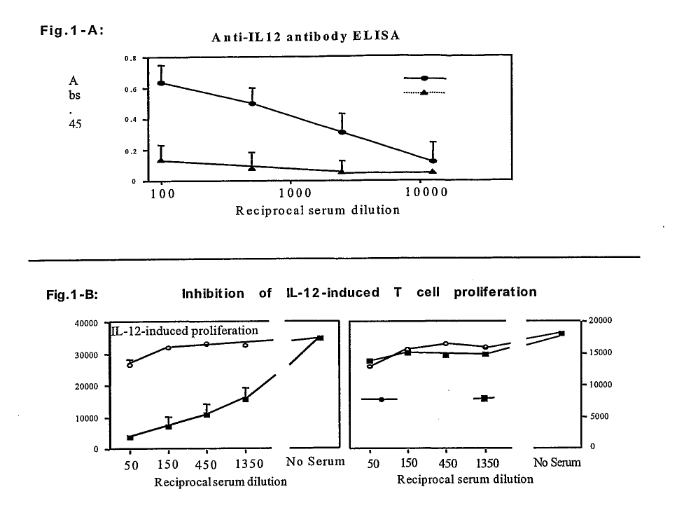

FIG. 1a shows results of C57Bl/6 mice immunized with IL-12-Ova, in the presence of an adjuvant comprising Liposome, 3D-MPL and QS21??.

FIG. 1b shows results of inhibition of IL-12 induced proliferation of ConA-activated T cells, in which Con-A blasts were incubated with IL-12 or IL-2 in the presence of control or anti-IL-12-Ova sera. After 48 h, thymidine incorporation was determined (mean±SEM for 5 mice/group.

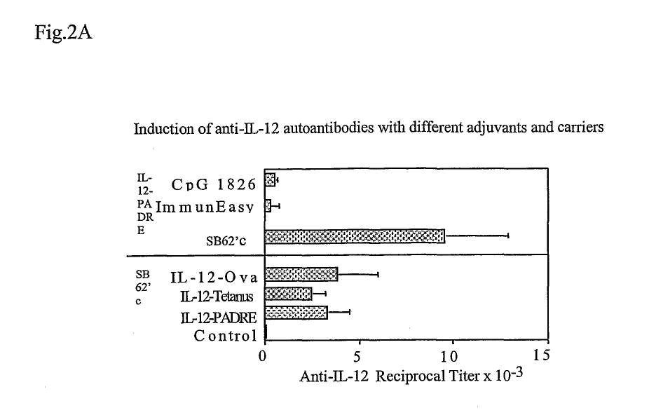

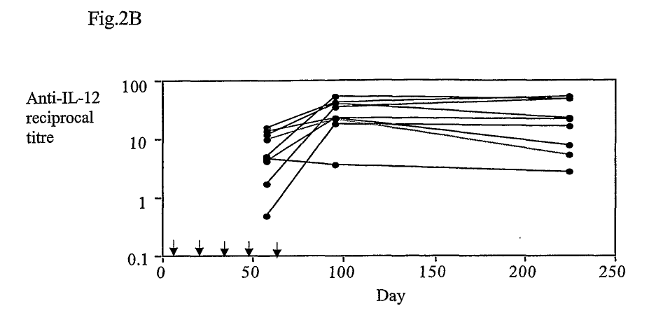

FIG. 2a shows C57Bl/6 mice immunized with IL-12 coupled to Ova or T-helper peptides (PADRE or Tetanus) in the presence of different adjuvants. IL-12 inhibitory activities were tested on IL-12-R transfected BaF3 cells.

FIG. 2b shows persistence of anti-IL-12 titers in the C57Bl/6 mice immunized with IL-12-PADRE complexes.

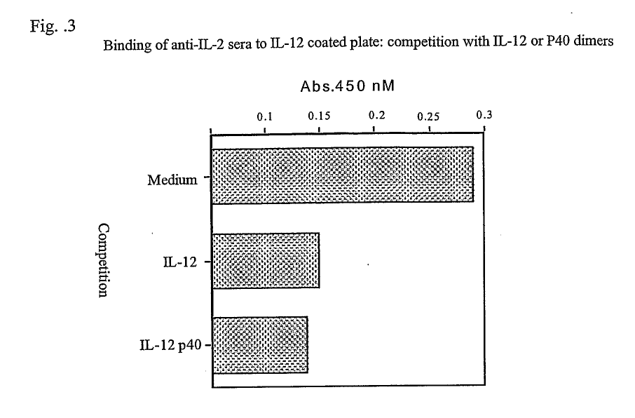

FIG. 3 shows sera from mice vaccinated with IL-12 PADRE complexes, preincubated with IL-12 heterodimer or IL-12 p40 homodimers before transfer to IL-12 coated plates. Boaund antibodies were detected using goat anti-mouse Ig.

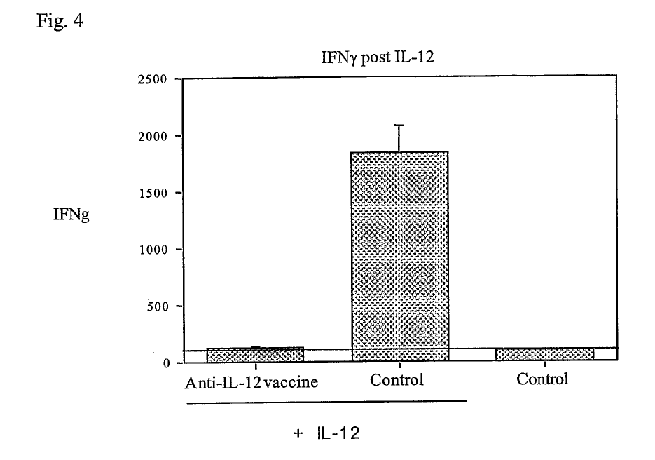

FIG. 4 shows Inhibition of IFNγ induction by IL-12 in anti-IL-12 vaccinated mice. C57Bl/6 mice vaccinated with IL-12-PADRE complexes in SB62′c adjuvant were treated with 500 ng IL-12 for 3 consecutive days. 24 h after the last injection, IFNγ concentrations were measured in the serum.

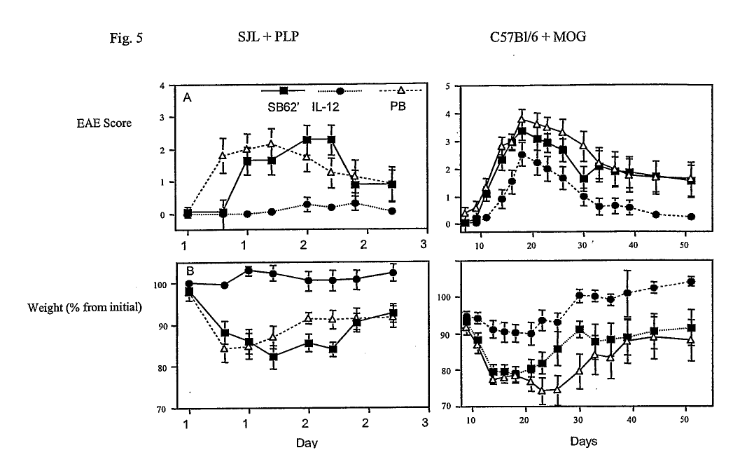

FIG. 5 shows reduced EAE severity in anti-IL-12 vaccinated mice. Groups of 13 SJL mice (A and B) previously vaccinated with IL-12-PADRE in AS2V or treated either with adjuvant only or PBS were immunized with PLP peptide for EAE induction. Similarly vaccinated of control groups of 15 C57Bl/6 mice (C and D) were treated with MOG encephalitogenic peptide. Mean EAE scores and body weights are shown. The differences in both readouts for SJL mice was highly significant (p<0.003 at any time point (Mann-Whitney)). For MOG-induced EAE, the differences in body weight were significant (p<0.5) at all time points except on day 26 (p=0.06). For MOG-induced-disease, EAE scores showed significant differences on days 11, 14 and from day 36 until the end of the experiment. Weight loss was significantly reduced on days 11, 14, 16, 18, 21, 23, 30, 33 and 36.

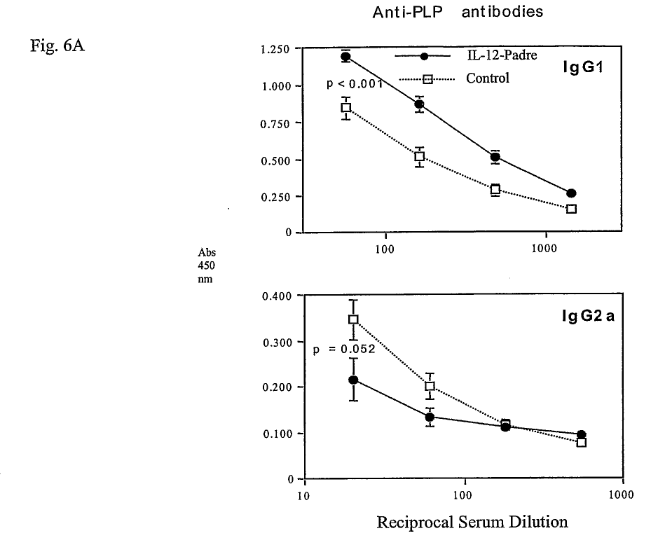

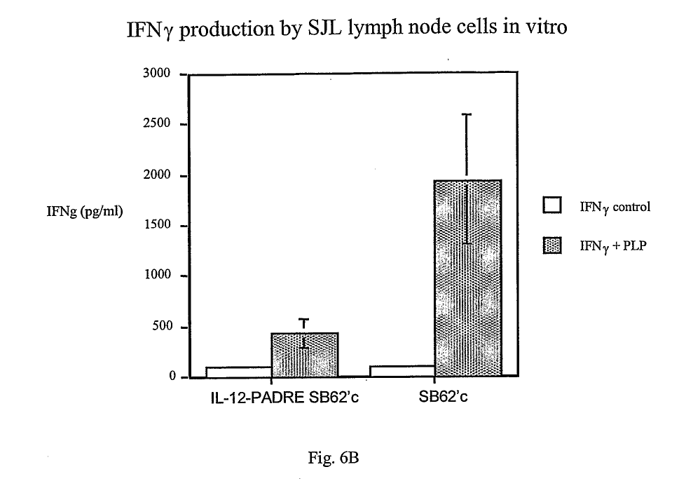

FIG. 6 shows detection of IgG1 and IgG2a anti-PLP antibodies. Serial dilutions of sera from SJL mice (12 mice/group) collected at termination of PLP-induced EAE in IL_{—}12-PADRE or vehicle+SB62′c vaccinated animals were incubated on PLP-coated plates. Bound antibodies were detected with subclass specific antibodies

### EXAMPLES

Material and Methods

### Example 1

Vaccine Preparation and Immunisation.

Mouse IL-12, histidine-tagged on p35, was prepared as described in Fallarino et al., JI, 1996 156(3): p.1095-1100] This product was coupled to Ova or helper peptides by overnight reaction under cooling with 20 mM glutaraldehyde in 0.1 M phosphate buffer at pH 6. The reaction was stopped by addition of Tris-HCl pH 9 (0.1 M final concentration) and the resulting products dialysed against PBS. For coupling to Ova, a 1/1 molar ratio per IL-12 subunit was used. Synthetic helper peptides selected for strong MHC Class II binding included Pan DR epitope peptide (PADRE) (aKXVAAWTLKAAC), and tetanus peptides (CQYIKANSKFIGITEL) or (cFNNFTVSFWLRVPKVSASHLE) [see: Alexander et al., Immunity, 1994. 1(9): p. 751-61]. These were coupled in ratios of 5 peptides per IL-12 subunit.

Other complexes were prepared by introducing sulfhydryl groups in IL-12 through reaction with 2-iminothiolane (Traut's reagent) before conjugation to maleimide-activated carriers, including Ova, keyhole limpet Haemocyanin (KLH) or cationised BSA according to the manufacturer protocols (Pierce, Ill., USA).

Vaccines were administered s-c or i.m. with one of the following adjuvants: complete Freund's adjuvant (CFA); Liposome/3D-MPL/QS21 (GSK); Immun-Easy Mouse Adjuvant (Qiagen, Valencia, Calif.); CpG oligodeoxynucleotide 1826 (5′-TCCATGACGTTCCTGACGTT-3′) with phosporothioate modification [Ballas et al., JI 2001 167(9) p4878-86]; and SB62′c, an adjuvant comprising 3D-MPL, an oil-in-water emulsion and a saponin, wherein the oil is a metabolisable oil, and the ratio of the metabolisable oil:saponin (w/w) is in the range of 1:1 to 200:1 (GSK, as described in WO99/11241, the full teaching of which is incorporated herein by reference).

### Example 2

Assessment of Anti-IL-12 Antibodies.

For detection of anti-IL-12 antibodies by ELISA, Maxisorb Nunc-Immunoplates (Nalge Nunc International, Hereford, U. K.) were coated with IL-12 or BSA as a control (both at 5 μg/ml) in 20 mM glycine buffer pH 9.3. After blocking with 1% BSA in PBS, sera diluted in blocking buffer were added to the plates and incubated at 37° C. for 2 h. After washing, peroxidase-coupled goat anti-mouse IgG (Transduction Laboratories, Lexington Ky.) followed with Ultra-TMB substrate (Pierce, Rockford, Ill., USA) were used to detect bound antibodies.

The specificity of these antisera was further analysed by pre-incubating appropriately diluted samples with IL-12 heterodimers or P40 homodimers (R&D, Minneapolis) both at 1 μg/ml for 2 h before incubation on IL-12-coated plates.

Inhibition of IL-12 activity was measured in vitro by testing inhibition of IL-12-induced proliferation of ConA-blasts prepared from C57Bl/6 spleen cells according to Schoenhaut [Schoenhaut et al., JI, 1992. 148(11) p3433-40] Alternatively, 10⁴ Baf3 cells transfected with murine IL-12 receptors (a kind gift of Dr. Jean-Christophe Renauld, LICR, Brussels Branch). were put in 96 well plates, in 200 μl DMEM with 10% FCS and proliferation was measured 48 h later after addition of tritiated thymidine for the last 16 hours. Inhibition titres were calculated as the reciprocal serum dilution giving 50% inhibition of 1 ng/ml IL-12.

### Example 3

Assessment of IL-12 Activity in Anti-IL-12 Immunised Mice In Vivo.

C57Bl/6 mice immunised with IL-12-PADRE or vehicle were treated on 3,consecutive days with 500 ng IL-12. One day after the last injection, blood was collected and IFNγ serum concentration was determined.

### Example 4

Induction of Experimental Allergic Encephalomyelitis (EAE).

EAE was induced in SJL and C57Bl/6 mice previously immunised with IL-12-PADRE complexes in an adjuvant comprising an oil-in-water emulsion and a saponin, wherein the oil is a metabolisable oil, and the ratio of the metabolisable oil:saponin (w/w) is in the range of 1:1 to 200:1 (GSK), or with adjuvant only. In SJL, EAE was elicited according to Weinberg [Weinberg, et al., JI, 1999. 162(3) p1818-26], using 150 μg proteolipid protein (PLP) peptide 139-151 (HCLGKWLGHPDKF) injected in CFA along with 200 μg Mycobacterium butyricum (Difco Lab., Detroit, Mich.) in 2×50 μl at the base of the tail and in 2×50 μl aliquots s.c. in the flanks. In C57Bl/6, 100 μg myelin oligodendrocyte glycoprotein (MOG) peptide 35-55 (MEVGWYRSPFSRVVHLYRNGK) was injected in CFA containing 800 μg Mycobacterium butyricum (2×50 μl sc at the base of the tail). Mice were then injected intravenously with 300 ng of Pertussis toxin (Calbiochem) in 100 μl PBS containing 1% NMS. The Pertussis toxin injection was repeated after 48 h according to the protocol described by Slavin [Slavin et al., Autoimmunity, 1998. 28(2) p109-20]. Disease was evaluated by determination of body weight and EAE scoring according to Heremans [Heremans, et al., Eur Cytokine Netw, 1999. 10(2) p171-80].

### Example 5

Determination of Antibody Responses to PLP Peptide.

Anti-PLP IgG1 and IgG2a antibodies were tested on Maxisorb plates coated with PLP peptide at 2 μg/ml. After blocking with 1% BSA, serial serum dilutions were incubated for 2 h and, after washing, anti-IgG1 (LOMG1) or anti-IgG2a (LOMG2a) rat antibodies coupled to HRP (IMEX, Brussels, Belgium) were added. Plates coated with BSA gave negligible signals.

Popliteal lymph nodes collected from 5 to 14 weeks after EAE induction were stimulated in vitro with PLP for 72 h and IFNγ was measured by ELISA (Biosource Europe Fleurus Belgium) or bioassay respectively.

### Example 6

ELISA

IFNγ concentrations in culture supernatant were determined by sandwich ELISA. Supernatants and appropriate cytokine standards (PharMingen, San Diego, Calif.) were used in threefold serial dilutions. Purified and biotinylated antibodies were purchased from PharMingen. Detection was performed with alkaline phosphatase-coupled streptavidin (Southern Biotechnology, Birmingham Ala.). Detection limits for IFNγ are 46 pg/ml. Serum samples and appropriate immunoglobulin standards (Southern Biotechnology, Birmingham, Ala.) were used in 3-fold serial dilutions. Detection limits were 5 ng/ml for IgG1 and 0.1 ng/ml for IgG2a. Total IgE was determined with mAbs 84.1C for coating and alkaline phosphatase labeled EM95.3 for detection. The detection limit for IgE was 10 ng/ml.

Results

### Example 8

Induction of Anti-IL-12 Auto-antibodies.

Immunisation of mice with mouse IL-9 coupled to Ova with glutaraldehyde and emulsified in CFA triggers the production of anti-IL-9 auto-antibodies, leading to efficient suppression of IL-9 activities in vivo [Richard, et al., PNAS USA, 2000. 97 p767-772.]. Similar attempts made with IL-12 were, however, not successful. We therefore changed the adjuvant to Liposome/3D-MPL/QS21 (GSK). This resulted, in C57Bl/6 mice, in the production of significant antibody titres as assessed by ELISA (FIG. 1A) and inhibition of IL-12-induced proliferation of ConA-activated T cells (FIG. 1B). The specificity of this inhibition was demonstrated by undiminished responses of similarly prepared blasts to IL-2.

These results highlighted the importance of the adjuvant for such immunisations. We therefore tested several other products with immune-stimulating properties, including SB62′c (GSK); ImmunEasy a commercial adjuvant based on CpG from Qiagen; and CpG 1826, a phosporothioate-modified DNA with CpG motifs. As shown in FIG. 2A, SB62′c induced responses that were approximately ten times better than those obtained with adjuvants not containing QS21 or 3D-MPL. In the same Figure are shown results obtained with IL-12 coupled to PADRE and Tetanus helper peptides. These complexes gave results essentially similar to those obtained with IL-12-Ova, indicating that an effective vaccine could be obtained by direct addition of the helper peptides.

Numerous methods, often more refined than that using glutaraldehyde, have been developed for protein cross-linking. One is to introduce free sulfhydryl groups in the protein of interest, which ensures its reaction with maleimide-substituted carriers. Such complexes were prepared with IL-12 by reacting the protein with Traut's reagent before cross-linking to maleimide-substituted Ova, KLH or cBSA. For comparison, mice were similarly immunised with IL12-OVA complexes made with glutaraldehyde. As shown in FIG. 2B, IL-12 coupled to Ova with both methods gave similar results. However, the other carriers were ineffective. These results prove that mere injection of IL-12 coupled to foreign carrier proteins, even with potent adjuvants, will not systematically break self-tolerance, but that proper combinations of carrier and adjuvant are required to induce significant responses.

Analysis of the kinetics of anti-IL-12 vaccination showed that neutralizing titers were observed only after multiple injections (usually 4 or 5), titers often continued to increase for several weeks after the last immunization and persisted for unlimited periods of time (FIG. 2C).

### Example 9

Specificity of Anti-IL-12 Antibodies

The complexes used for immunisation were made with recombinant IL-12p70 (p40-p35 heterodimers). Since the antisera showed antibody binding to IL-12 p70 coated plates, competition experiments were carried out to analyse their relative interactions with p40 versus p70. Appropriately diluted sera were incubated with IL-12 p70 or p40 homodimers prior to transfer to IL-12-coated plates. Both P40 dimers and IL-12 heterodimers had equivalent inhibitory activities, indicating that most of the anti-IL-12 antibodies reacted with the p40 subunit. (FIG. 3).

### Example 10

Anti-IL-12 Vaccinated Mice No Longer Respond to IL-12 In Vivo.

Repeated administration of IL-12 to normal mice induces elevated IFNγ levels in the serum [Gately, et al., Int Immunol, 1994 6(1) p157-67]. We used this procedure to evaluate the functional efficacy of anti-IL-12 vaccination. As shown in FIG. 4, after injection of IL-12 for 3 consecutive days, IFNγ levels were in the nanogram/ml range in control mice but remained undetectable (<0.03 ng/ml) in anti-IL-12 -vaccinated animals.

### Example 11

Anti-IL-12 Vaccine Impairs EAE-induction.

SJL mice were immunized with IL-12-PADRE peptides or vehicle in the presence of SB62′c adjuvant before induction of EAE by immunization with PLP peptide. After four injections, reciprocal anti-IL-12 neutralizing antibody titers were 6,513±2,012. As shown in FIG. 5, EAE symptoms became apparent in control adjuvant-treated mice from day 12, peaked around day 20 (one of the animals died on day 17), then gradually subsided but were still detectable after one month in one third of the animals. In anti-IL-12 vaccinated mice only minimal signs of disease were detected and all mice survived. Moreover, body weight drop, another feature of PLP-induced EAE, was completely absent in the vaccinated animals. Of note, administration of SB62′c by itself had a slight protective activity as compared to mice receiving simply PBS.

The protective effect of IL-12 vaccination was expected to imply suppression of IFNγ production and changes in anti-PLP antibody IgG subclasses.

Analysis of anti-PLP IgG1 and IgG2a antibodies, showed that there was a clear increase in IgG1 anti-PLP titres (p<0.001) and a reduction in IgG2a that was at the limit of statistical significance (p=0.052) (FIG. 6A). Together, these results clearly show that IL-12 vaccination induces fundamental changes in anti-PLP response.

The former hypothesis was tested with lymph node cells stimulated in vitro with PLP peptide. IFNγ concentrations were 430±139 pg/ml in 8 IL-12 vaccinated mice and 1939±634 in 9 SB62′c controls (p=0.0079 Mann-Whitney). Popliteal lymph nodes collected from 5 to 14 weeks after EAE induction (8 and 9 mice in IL-12-PADRE and SB62′c groups) were stimulated in vitro with PLP peptide. IFNγ concentrations were measured after 3 days (FIG. 6B).

To test whether anti-IL-12 vaccination would also prevent the more aggressive form of EAE induced by immunisation with MOG peptide, C57Bl/6 mice vaccinated with IL-12-PADRE complexes in the presence of SB62′c before immunisation with MOG had reciprocal inhibition titres of 19,577±3,792. Extremely elevated EAE scores were noted in the control group and 2 of the 15 mice in this population died after 26 and 33 days respectively, Anti-IL-12 vaccinated mice showed a 2-3 day delayed onset and reduced maximal disease scores as well as body weight losses. Moreover, none of these mice died and 11/15 showed complete recovery, which occurred only in 4/15 controls (p=0.027 by Fisher's statistics). Also in MOG-induced EAE was there a protective effect of SB62′c as compared to PBS-treated mice. This was particularly striking for body weight recovery, which was accelerated by more than a week.

To further evaluate the potency of our vaccine and to compare it with results obtained by administration of anti-IL-12 antibodies, one additional groups was included in the former MOG experiment. This group received repeated injections of C17.8, a rat anti-p40 antibody, which has previously been shown to inhibit EAE in NOD mice [Ichikawa et al., J Neuroimmunol, 2000. 102(1) p56-66]. As shown in Table 1, mean weight losses and EAE scores in C57Bl/6 mice were reduced by these antibodies to similar levels as those observed with the IL-12-PADRE vaccine. The figures correspond to 14 measurements made from day 9 to day 51 in 15 C57Bl/6 mice per group. The probabilities were calculated by Mann-Whitney non-parametric statistics.

TABLE 1

| | Weight | P | EAE score | P |
|---|---|---|---|---|
| IL-12-PADRE- | 95 +/− 1.68 | | 1.028 +/− 0.229 | |
| SB62′c | | | | |
| SB62′c | 85.6 +/− 9 | 0.0045 | 2.086 +/− 0.33 | 0.023 |
| C17.8 | 90.53 +/− 1.94 | | 1.16 +/− 0.138 | |
| PBS | 81.6 +/− 3.12 | 0.0094 | 2.257 +/− 0.357 | 0.009 |

1. An immunogenic composition comprising:
(a) an immunogen comprising
(i) IL-12, IL-23, or a subunit or component thereof; and
(ii) a carrier; and
(b) an adjuvant comprising one or more of cholesterol; oil-in-water emulsion; oil-in-water emulsion low dose; tocopherol; liposome; QS21; and 3D-MPL.

2. The immunogenic composition according to claim 1 in which the immunogen comprises the P35 subunit of IL-12.

3. The immunogenic composition according to claim 1 in which the immunogen comprises the P40 subunit of IL-12 or IL-23.

4. The immunogenic composition according to claim 2 in which the immunogen comprises at least one surface epitope of P35 or P40.

5. The immunogenic composition according to claim 1 in which the carrier comprises one or more of: Keyhole Limpet Haemocyanin (KLH); bovine serum albumin (BSA); tetanus toxin (TT), diphtheria toxin (DT); Domain 1 of Fragment C of TT; the translocation domain of DT; Hep B core protein; PADRE; P2; and P30.

6. The immunogenic composition according to claim 1 in which component (i) is coupled to the carrier by direct covalent coupling.

7. The immunogenic composition according to claim 1 in which component (i) is fused to the carrier.

8. The immunogenic composition according to claim 1 in which the adjuvant comprises liposome, 3D-MPL and QS21.

9. The immunogenic composition according to claim 1 in which the adjuvant comprises oil-in-water emulsion low dose; 3D-MPL and QS21.

10. The immunogenic composition according to claim 1 in which the adjuvant comprises oil-in-water emulsion low dose; 3D-MPL and QS21.

11. The immunogenic composition according to claim 1, in which the adjuvant comprises oil-in-water emulsion.

12. The process for the manufacture of an immunogenic composition according to claim 1 comprising mixing immunogen (a) with the adjuvant.

13. The vaccine composition comprising the immunogenic composition as described in claim 1 in combination with a pharmaceutically acceptable excipient, adjuvant or vehicle.

14. A process for making the vaccine composition according to claim 13 comprising mixing an immunogenic composition comprising:
(a) an immunogen comprising
(i) IL-12, IL-23, or a subunit or component thereof; and
(ii) a carrier: and
(b) an adjuvant comprising one or more of cholesterol: oil-in-water emulsion; oil-in-water emulsion low dose; tocopherol; liposome; QS21; and 3D-MPL with a pharmaceutically acceptable excipient, adjuvant or vehicle.

15. The method of preventing or treating a disease or disorder, in particular an autoimmune-implicated disease by administration of an immunogenic composition according to claim 1.

16. (canceled)

17. The method according to claim 15, in which the composition is for prevention, therapy or treatment of a disease or disorder of a mammal.

18. The method according to claim 15, in which the composition is for prevention, therapy or treatment of a disease or disorder of a human.

19. The method according to claim 15, in which the composition is for prevention, therapy or treatment of a disorder chosen from the group of: multiple sclerosis; Crohn's disease; thyroiditis; and rheumatoid arthritis.

20. A kit comprising an immunogen according to any preceding claim and an adjuvant comprising one or more of cholesterol; oil-in-water emulsion; oil-in-water emulsion low dose; tocopherol; liposome; QS21; and 3D-MPL.
